(19) United States
(12) Patent Application Publication
(10) Pub. No.: US 2023/0420140 A1
(43) Pub. Date: Dec. 28, 2023

(54) SYSTEM AND METHOD FOR PROCESSING GLUCOSE DATA

(71) Applicant: Laxmi Therapeutic Devices, Inc., Goleta, CA (US)

(72) Inventors: Jason GOLDBERG, Oakland, CA (US); William VAN ANTWERP, Santa Clarita, CA (US)

(21) Appl. No.: 18/151,396

(22) Filed: Jan. 6, 2023

Related U.S. Application Data
(60) Provisional application No. 63/354,659, filed on Jun. 22, 2022.

(51) Int. Cl. G16H 50/30

(52) U.S. Cl. CPC G16H 50/30

(57) ABSTRACT

A system and method for processing glucose data. In some embodiments, the method includes estimating the severity of diabetes in a subject. The estimating may include comparing distributional glucose data of the subject, and distributional glucose data of one or more reference subjects.

### CROSS-REFERENCE TO RELATED APPLICATION(S)

The present application claims priority to and the benefit of U.S. Provisional Application No. 63/354,659, filed Jun. 22, 2022, entitled “SYSTEM AND METHOD FOR PROCESSING GLUCOSE DATA”, the entire content of which is incorporated herein by reference.

### FIELD

One or more aspects of embodiments according to the present disclosure relate to analysis of health data, and more particularly to a system and method for processing glucose data.

### BACKGROUND

Type 2 diabetes and pre-diabetes are a large and growing health problem. In the United States there are 37.3 million people with diabetes. 1.9 million have Type 1 diabetes, 35.4 million have Type 2 diabetes (8.5 million undiagnosed) and more than 96 million (nearly 30% of all Americans) have pre-diabetes. But these numbers (except for the Type 1 numbers) are just estimates, based on some defined but not absolute criteria.

According to the National Institute of Diabetes and Digestive and Kidney Diseases, hemoglobin A1C (HbA1c) less than 5.7% is normal, 5.7-6.4% is pre-diabetes, and >6.4% is Type 2 diabetes. HbA1c is a measure of glucose exposure over the course of about the previous 6-weeks. Similarly, a fasting plasma glucose of less than 100 mg/dl is normal, 100-125 mg/dl indicates pre-diabetes and 126 or higher is indicative of Type 2 diabetes. Alternatively, an oral glucose tolerance test (OGTT) (75 g glucose) of less than 140 mg/dl (at 2 hours) is normal, 140 to 199 mg/dl is indicative of pre-diabetes and more than 199 mg/dl indicates Type 2 diabetes. The World Health Organization has defined pre-diabetes as fasting glucose of between 110 and 125 mg/dl.

While these definitions appear to be reasonable, they do not consistently agree with each other. Several studies have shown that the correlation among the various diagnostic parameters is very poor. In a recent study data was obtained from a cohort of nominally healthy individuals (n=57). Baseline data (HbA1c, fasting plasma glucose (FPG), OGTT, insulin secretion rate and others) were obtained together with a series of continuous glucose monitor traces from daily life and from several controlled meals.

In the data, none of the variables associated with diagnosis of pre-diabetes or Type 2 diabetes have correlations greater than 0.65 with each other. Several of the patients in the study would be classified as having Type 2 diabetes by one measure, and as being non-diabetic (healthy) by the other 2-methods. Since the typical physician will only use one metric, diagnosis of glycemic health may often be incorrect.

It is with respect to this general technical environment that aspects of the present disclosure are related.

### SUMMARY

According to an embodiment of the present disclosure, there is provided a method, including: estimating the severity of diabetes in a subject, the estimating including comparing: distributional glucose data of the subject, and distributional glucose data of one or more reference subjects.

In some embodiments, the comparing includes calculating a measure of distance between the distributional glucose data of the subject, and the distributional glucose data of the one or more reference subjects.

In some embodiments, the measure of distance is a Wasserstein distance.

In some embodiments, the measure of distance is a Cramer distance.

In some embodiments, the measure of distance is a Jensen-Shannon distance.

In some embodiments, the distributional glucose data of the subject is based on a plurality of glucose measurements taken at different points in time.

In some embodiments, the distributional glucose data of the subject includes an estimated probability function of a glucose level of the subject.

In some embodiments, the estimated probability function is a kernel density estimate based on the distributional glucose data.

In some embodiments, the glucose level is an interstitial glucose concentration of the subject.

In some embodiments, the glucose level is a blood glucose concentration of the subject.

In some embodiments, the distributional glucose data of the subject includes a set of ordered pairs, each ordered pair including a glucose measurement taken at a respective first point in time, and a glucose measurement taken at a point in time separated from the first point in time by a fixed time interval.

In some embodiments, the fixed time interval is within 50% of 60 minutes.

In some embodiments, the distributional glucose data of the subject includes an estimated multi-variate probability density function of a glucose level of the subject.

In some embodiments, the distributional glucose data of the subject includes a Fourier transform of the plurality of glucose measurements.

In some embodiments, the one or more reference subjects include a subject diagnosed with prediabetes.

In some embodiments, the one or more reference subjects include a subject diagnosed with Type 2 diabetes.

According to an embodiment of the present disclosure, there is provided a system, including: a processing circuit; and memory, operatively connected to the processing circuit and storing instructions that, when executed by the processing circuit, cause the system to perform a method, the method including: estimating the severity of diabetes in a subject, the estimating including comparing: distributional glucose data of the subject, and distributional glucose data of one or more reference subjects.

In some embodiments, the comparing includes calculating a measure of distance between the distributional glucose data of the subject, and the distributional glucose data of the one or more reference subjects.

In some embodiments, the distributional glucose data of the subject is based on a plurality of glucose measurements taken at different points in time.

In some embodiments, the distributional glucose data of the subject includes an estimated probability function of a glucose level of the subject.

### BRIEF DESCRIPTION OF THE DRAWINGS

These and other features and advantages of the present disclosure will be appreciated and understood with reference to the specification, claims, and appended drawings wherein:

FIG. 1A is a graph of estimated probability density functions (PDFs), according to an embodiment of the present disclosure;

FIG. 1B is a graph of estimated cumulative distribution functions (CDFs), according to an embodiment of the present disclosure;

FIG. 2A is a graph of estimated probability density functions (PDFs), according to an embodiment of the present disclosure;

FIG. 2B is a graph of estimated cumulative distribution functions (CDFs), according to an embodiment of the present disclosure;

FIG. 3 is a graph of Cramer distances, according to an embodiment of the present disclosure;

FIG. 4 is a graph of Cramer distances, according to an embodiment of the present disclosure;

FIG. 5 is a graph of health scores, according to an embodiment of the present disclosure;

FIG. 6 is a graph of health scores, according to an embodiment of the present disclosure;

FIG. 7 is a table of Wasserstein distances, according to an embodiment of the present disclosure;

FIG. 8 is a Poincaré plot is a graph of health scores, according to an embodiment of the present disclosure;

FIG. 9 is a Poincaré plot is a graph of health scores, according to an embodiment of the present disclosure;

FIG. 10 is contour plot of a bivariate probability density function, according to an embodiment of the present disclosure; and

FIG. 11 is contour plot of a bivariate probability density function, according to an embodiment of the present disclosure.

### DETAILED DESCRIPTION

The detailed description set forth below in connection with the appended drawings is intended as a description of exemplary embodiments of a system and method for processing glucose data provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

To perform processing and analysis of glucose data, some embodiments use the distribution of glucose profiles (e.g., their probability density functions) and attempt to associate them with clinical outcomes. In some embodiments, distributional glucose data are used, for example, to estimate the severity of diabetes in a subject. As used herein, “distributional data” is a representation of how the relative proportions of glucose data are spread over some distribution domain such as signal amplitude or signal frequency.

Among those at risk for Type 2 Diabetes (T2D), continuous glucose monitors (CGMs) may be used to provide insight into the glycemic implications of food and lifestyle choice. CGMs may measure interstitial glucose concentration or blood glucose concentration. Machine learning (ML) techniques may be used to make inferences about glycemic health from CGM data. For example, supervised machine learning methods may use data derived from subjects of a priori-known health status to train a learning algorithm which, in turn, could accept new test subject data to make inferences about the health status of such new subjects. As another example, unsupervised learning of data (using, e.g., a clustering method) into groups (e.g., two groups, corresponding to nondiabetic and Type 2 diabetic subjects respectively) may be used.

CGMs may be used to estimate HbA1C and to distinguish, using methods disclosed herein, between prediabetes (PD) and Type 2 Diabetes. In some embodiments, ML methods are applied to CGM data in order to track glycemic health status over time. A family of numerical metrics or scores may be employed to quantify glycemic health along a continuum extending from nondiabetic subjects to subjects with Type 2 diabetes. For example, a subject's CGM data over a time window may be represented as an estimated probability function. Examples include the probability density function (PDF) or the cumulative distribution function (CDF) of glucose concentration or measures of glucose dynamics (e.g., changes in glucose, time derivatives, or lagged glucose). Each of the PD F and the CDF is an example of distributional data as that term is used herein. Statistical distances may be computed between a subject's PDF and reference PDFs from a large number of training subjects with known health status (e.g., nondiabetic (ND), prediabetic, and Type 2 diabetic). These distances may be combined to produce a single numerical score, which may be an estimate of the severity of diabetes in a subject (with the lowest severity corresponding to a nondiabetic subject). This score may be tracked overtime to quantify changes in glycemic wellness and provide earlier indication of improving or worsening health status.

Each subject's CGM data may take the form of a uniformly sampled glucose concentration time-series:

gₖ[mg/dl] k∈

[symbol]

where k is the sample index over the set of natural numbers [symbol] . Derivatives, e.g., nth order derivatives, of glucose g⁽ⁿ⁾ₖ may be estimated using a variety of methods (e.g., Savitsky-Golay filtering). Changes in glucose over a D sample delay may be denoted as:

Δₖ=gₖ−g_{k-D}.

A column vector of observations associated with sample index k may be denoted as xₖ. Examples include:
xₖ=[gₖ] glucose alone
xₖ=[gₖ,Δₖ]^{T} glucose and change in glucose from D samples earlier
xₖ=[gₖ,g_{k-D}]^{T} glucose and D-sample lagged glucose, where [.]^{T} is vector transpose.

For a length L window of samples spanning contiguous sample indices:

k∈ [symbol] ={k+ [symbol] , . . . , k+ [symbol] +L−1},

an estimated CDF over this window may be denoted as [symbol] (x). Techniques such as kernel density estimation (KDE) may be used to estimate [symbol] (x). Alternatively, if a good parametric description of the data is available (e.g., log-normal glucose), the unknown distribution parameters may be estimated using techniques such as Maximum Likelihood Estimation (MLE).

The reference health status indices may be labeled as j=0, 1, 2 for the nondiabetic, prediabetic, and Type 2 diabetic categories, respectively, and the number of reference subjects in each category may be denoted as Np. The estimated CDF fo the ith reference subject in category j may be denoted as G_{j,i}(x). Alternatively, a single composite CDF per-health-status may be computed, and may be denoted as \tilde{G}(x). The use of full CDFs may be contrasted with more limited, scalar glycemic health indicators derivable from the CDF, like median and Time-in-Range. In this sense, the CDF represents a super-set of such scalar glycemic health indicator metrics.

Statistical distance metrics may be employed to quantify the difference between two generally multi-dimensional random variables in terms of their PDFs or CDFs. Such distances may be used to make inferences about glycemic health. They may each possess certain convenient properties of distance metrics (e.g., non-negativity, identity of indiscernible elements, symmetry, and the triangle inequality). One family of distance metrics between CDFs F(x) and G(x) is the p-th order Cramer distance, e.g.,

d=(∫|F(x)−G(x)|^{p}dx)^{1/p}. (1)

The p-th order distances between [symbol] (x) (the CDF for the subject of interest over a time window with index [symbol] ) and the CDF of the ith reference subject in category j may be denoted as [symbol] where, again, j is the health category index, and i is the reference subject index. Other metrics (e.g., the Jensen-Shannon distance, or the Wasserstein distance) may be used instead of the Cramer distance.

Such statistical differences may be used in various ways to produce a numerical health score, which may be an estimate of the severity of diabetes in a subject. One such health score is that of average distance from nondiabetic references:

$${s}_{ℓ} = \frac{1}{{N}_{0}} {\sum }_{i = 0}^{{N}_{0} - 1} {d}_{ℓ , 0 , i} . \qquad ( 2 )$$

When the subject's distribution is close to (or far from) the nondiabetic reference distributions, such a score may be low (or high). Alternatively, scores measuring distances from references in multiple health status categories may be calculated.

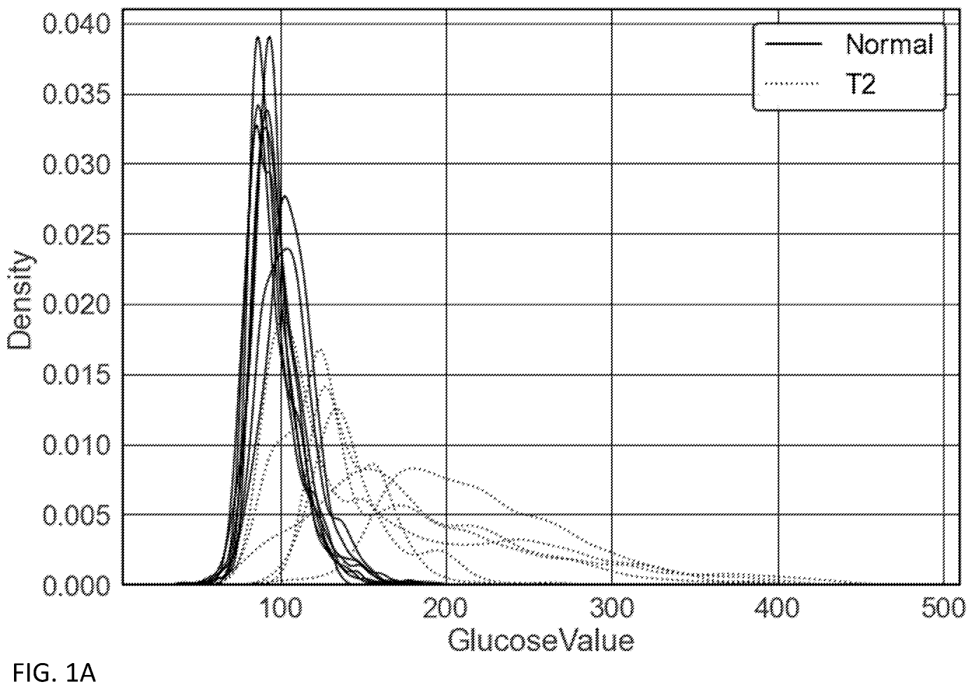

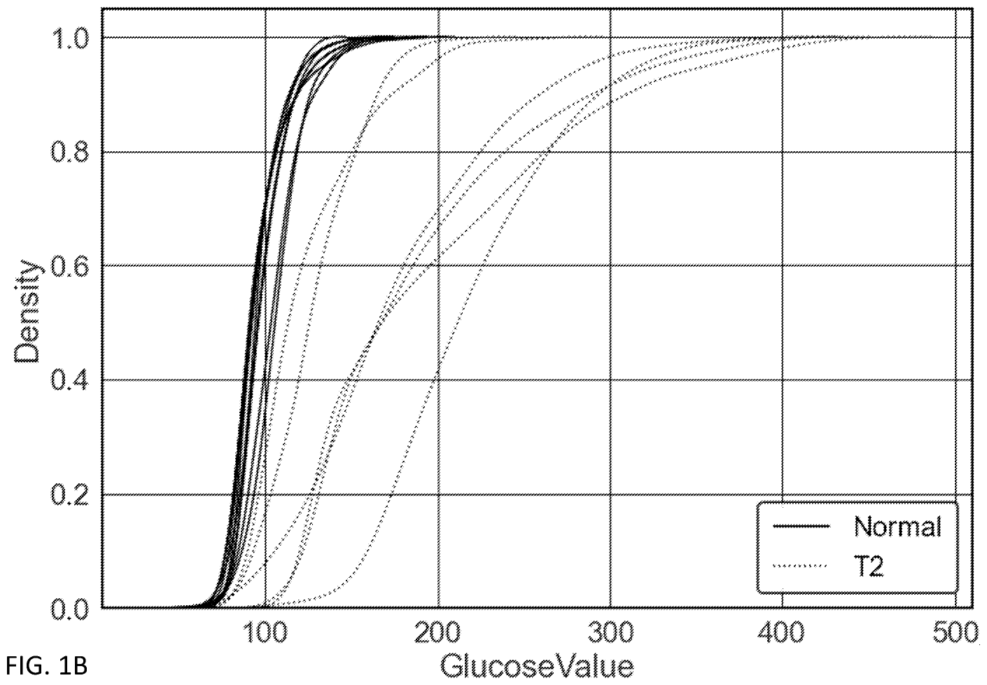

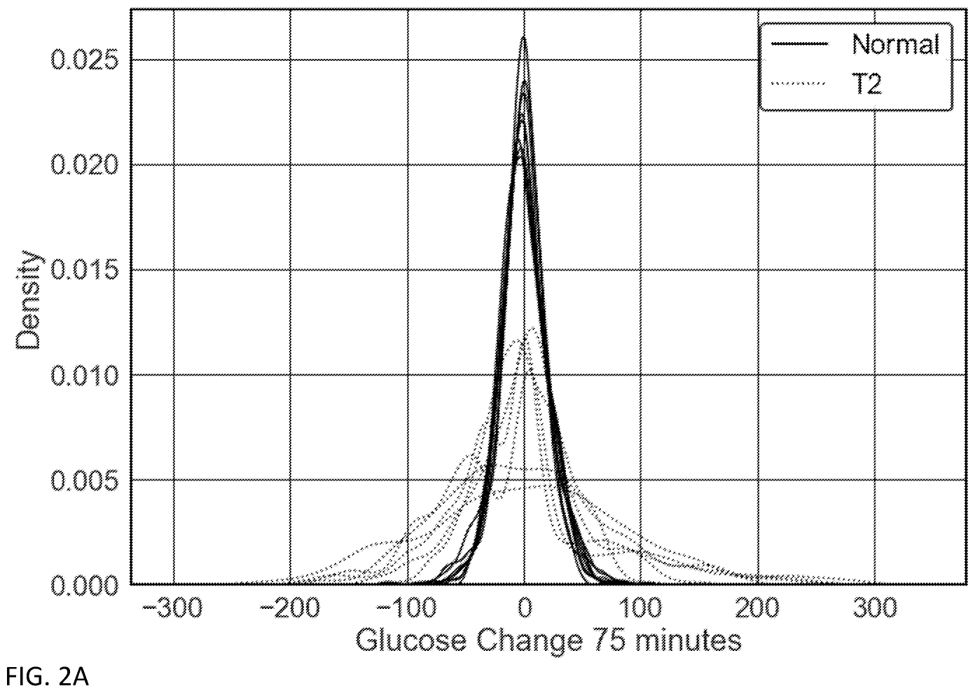

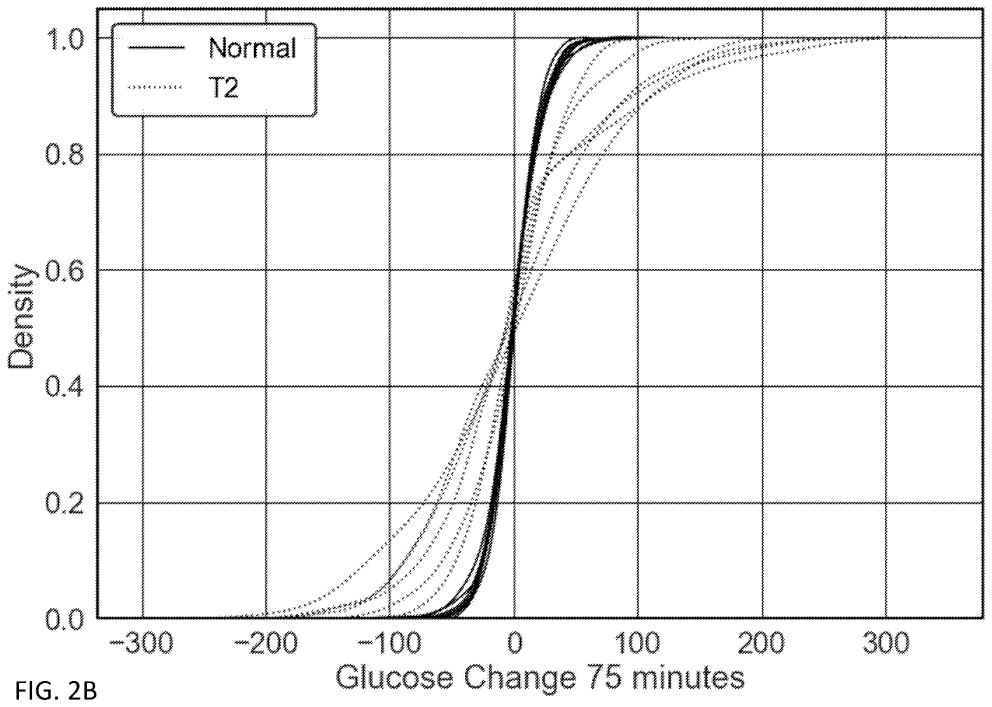

As an example of a reduction to practice, clinical trial CGM time-series data (with a five-minute sample interval) from ten nondiabetic subjects and ten Type 2 diabetic subjects were analyzed. FIGS. 1A and 1B show KDE-type estimates of PDF and CDF respectively, for glucose xₖ=[gₖ], for the nondiabetic and Type 2 diabetic subjects. FIGS. 2A and 2B show KDE-type estimates of PDF and CDF respectively, for 60-minute change in glucose xₖ [Δₖ], for the nondiabetic and Type 2 diabetic subjects. The figures indicate significant differences between nondiabetic and Type 2 diabetic subjects-especially for glucose level (FIGS. 1A and 1B). The plots also show more heterogeneity among the Type 2 diabetic subjects than among the nondiabetic subjects.

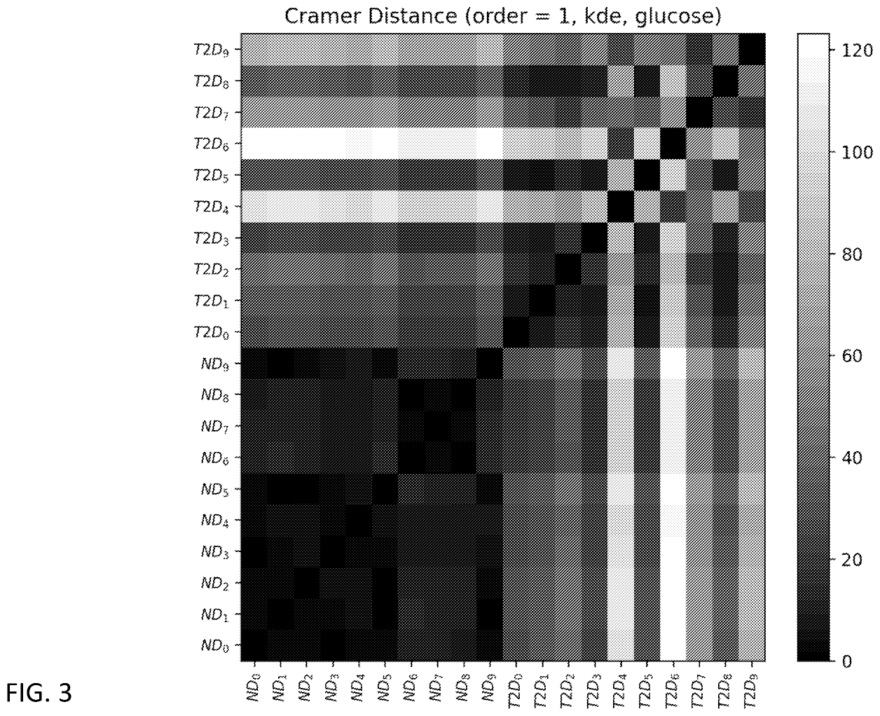

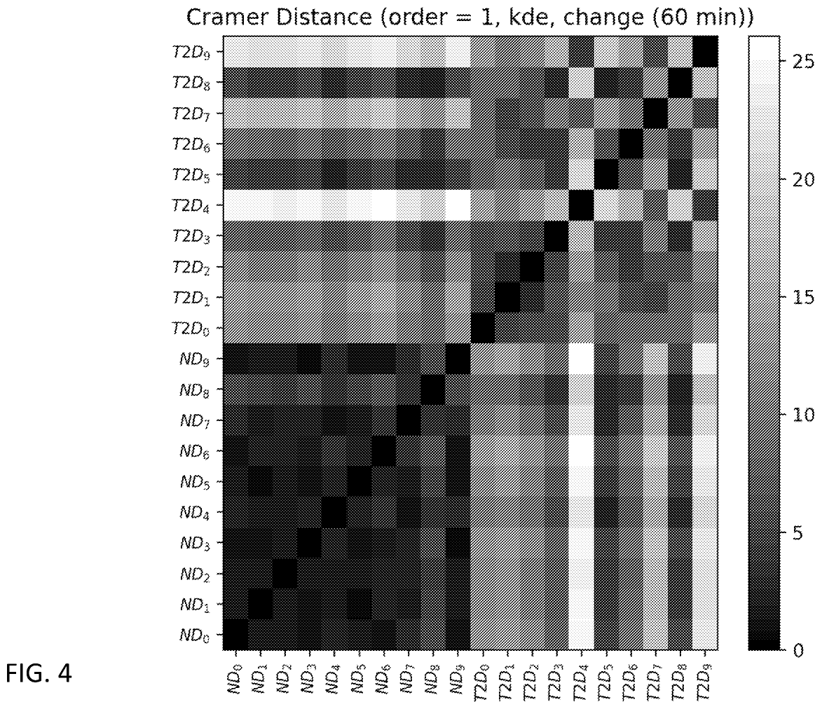

FIG. 3 shows pairwise first order (p=1) inter-subject Cramer distances (calculated according to Equation (1)) between the CDFs illustrated in FIG. 1B. FIG. 4 similarly shows pairwise first order (p=1) inter-subject Cramer distances between the CDFs illustrated in FIG. 2B. In FIGS. 3 and 4, the individual nondiabetic and Type 2 diabetic subject indices are denoted as NDᵢ and T2Dᵢ∈{0, 1, . . . , 9}, respectively. FIGS. 3 and 4 indicate generally relatively low inter-subject distances between pairs of nondiabetic subjects and generally a relatively high inter-subject distance between any nondiabetic subject and any Type 2 diabetic subject. Again, there appears to be more variability in distances between CDFs of Type 2 diabetic subjects than in distances between CDFs of nondiabetic subjects. FIGS. 3 and 4 also suggest that the differences in distance between nondiabetic subjects and Type 2 diabetic subjects are more pronounced for glucose than for glucose change.

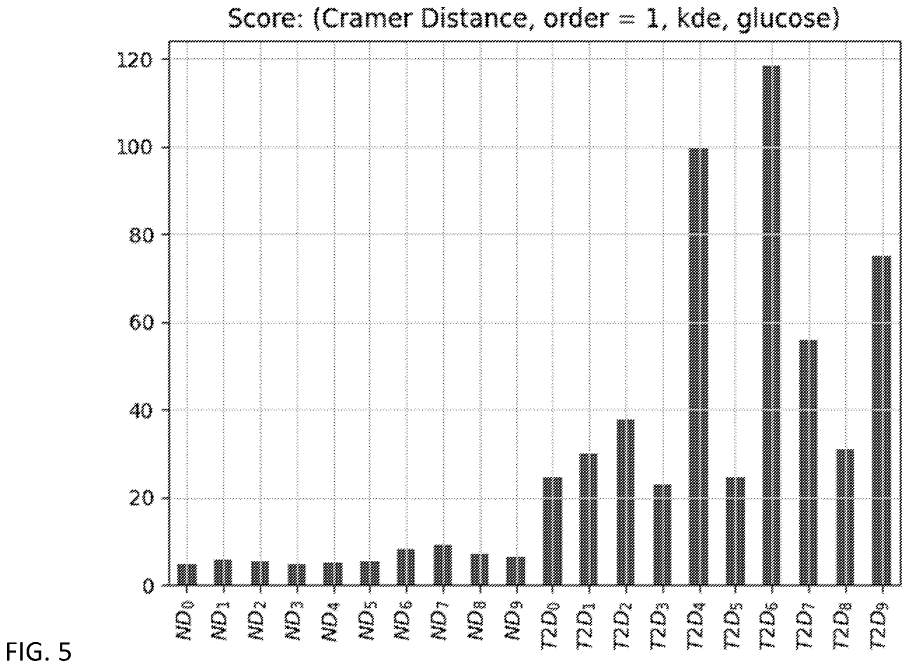

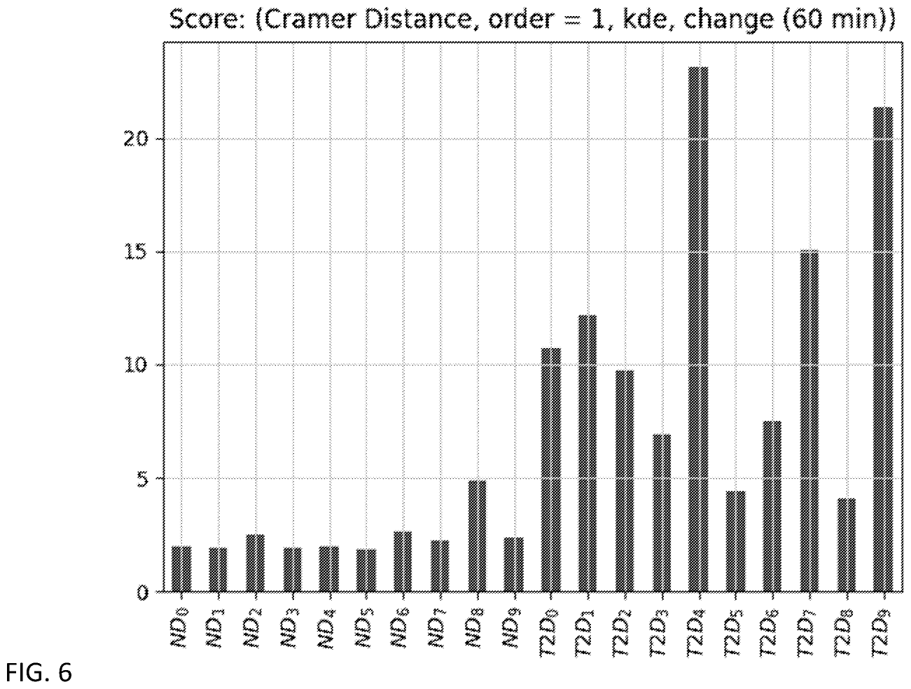

Health scores calculated according to Equation (2) are shown in FIGS. 5 and 6. Score computation for the ith nondiabetic subject omits the zero-distance term between the ith nondiabetic subject and itself in the averaging of Equation (2). As expected, the figures show generally lower scores for nondiabetic subjects than for Type 2 diabetic subjects. Again, the contrast is more pronounced for glucose as opposed to change in glucose over 60 minutes (with, for example subject T2D₈ having a lower score, in FIG. 6, than subject ND₈).

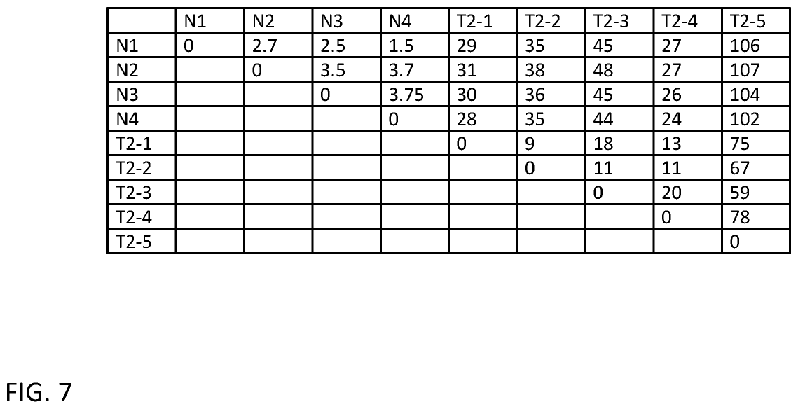

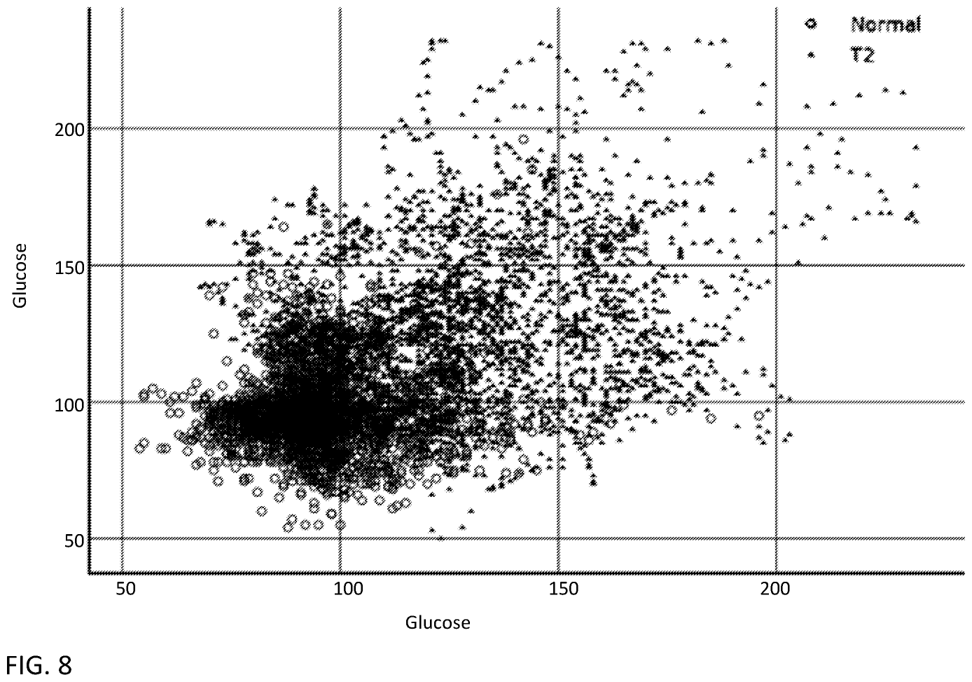

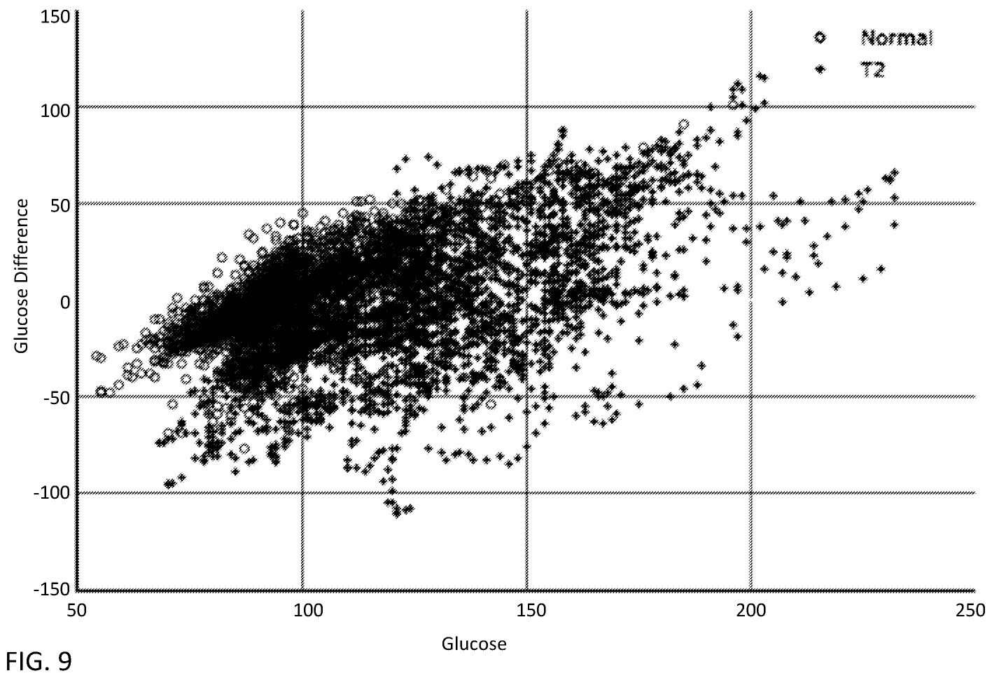

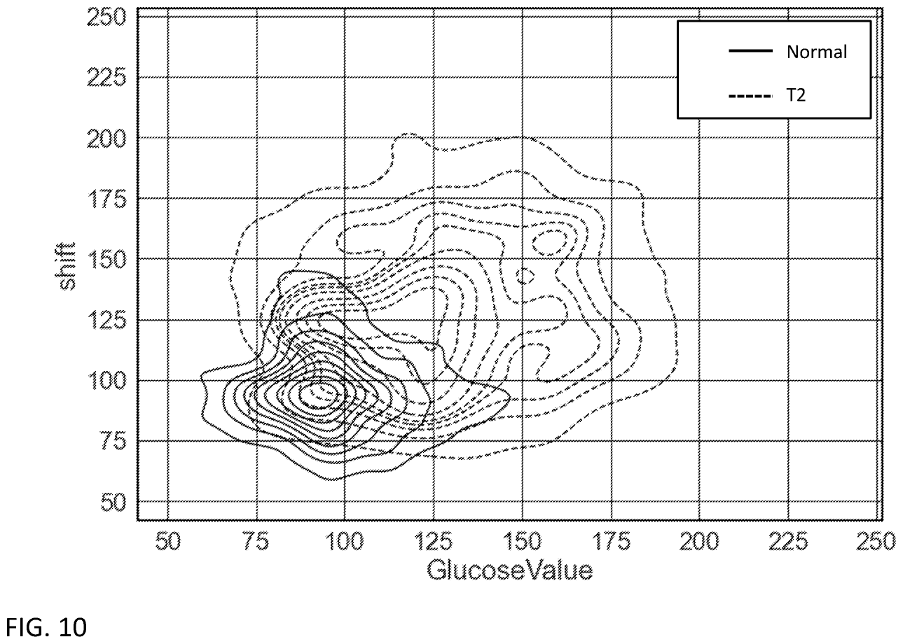

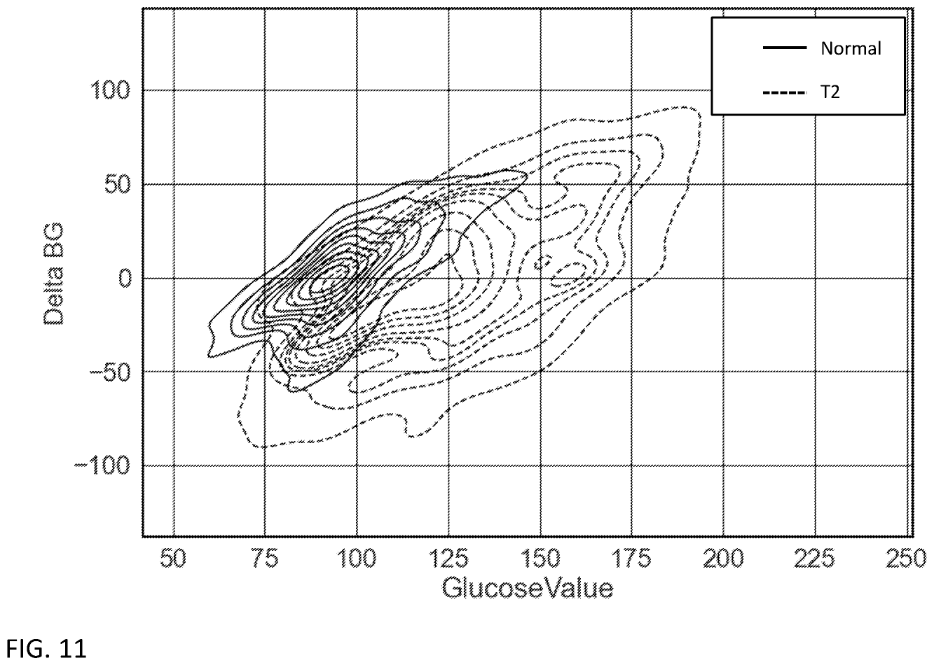

FIG. 7 is a table of Wasserstein distances for estimated PDFs (using KDE) for four nondiabetic subjects and five Type 2 diabetic subjects. It may be seen that the distances between nondiabetic subjects are less than 4 mg/dl whereas the distance between each Type 2 diabetic subject and any nondiabetic subject is at least 9, and most of these differences are significantly larger. FIG. 8 is a Poincaré plot for a nondiabetic subject and a Type 2 diabetic subject. The Poincaré plot uses the measured blood glucose on the X axis and the measured blood glucose after a time interval of 60 minutes on the Y axis. FIG. 9 is also a Poincaré plot for a nondiabetic subject and a Type 2 diabetic subject. The Poincaré plot of FIG. 9 uses the measured blood glucose on the X axis and the difference between consecutive measured blood glucose values on the Y axis. FIG. 10 is a contour plot of a bivariate KDE (a kernel density estimate of a bivariate PDF) for a nondiabetic subject and a bivariate KDE for a Type 2 diabetic subject, with the variable corresponding to the X axis being the measured blood glucose and the variable corresponding to the Y axis (the “shift”) being the measured blood glucose after a time interval of 60 minutes. FIG. 11 is a contour plot of a bivariate KDE for a nondiabetic subject and a bivariate KDE for a Type 2 diabetic subject, with the variable corresponding to the X axis being the measured blood glucose and the variable corresponding to the Y axis (the “Delta BG”) being the difference between consecutive measured blood glucose values.

Specific examples of methods for analyzing health data (e.g., glucose data) are described above, but the present disclosure is not limited to these specific examples. For example, some methods may use, as distributional data, probability functions based on transformed versions of the glucose time series (e.g., based on a logarithm of glucose rather than glucose itself, or on a Fourier transform (e.g., a fast Fourier transform (FFT)) of the glucose signal). Scores incorporating temporal weighting of data (e.g., less weight during quiescent overnight periods) may be used. Other forms of weighting (beyond temporal weighting) may be used. For example, a weighting function may be included inside the integral expression for the Cramer distance that would emphasize or deemphasize contributions to the integral at different signal amplitudes. Scores based on ambulatory glucose profile (with or without averaging over time) may be used. Distances based on parametric or non-parametric modeling of, for example, glucose level (e.g., log-normal) may be used. Distances based on simple first and second order statistics (e.g., a Wasserstein-2 formula even for non-Gaussian measurements, for which a closed-form expression may not exist) may be used. Distances based on a probability function associated with parametric or non-parametric modeling of meal response (e.g., for a controlled meal) (e.g., a joint PDF of meal response height and width) may be used.

Distances based on statistical quantities other than probability functions (e.g., temporal correlation/covariance over many lags, power spectrum) may be used. Distance metrics which exploit the quasi-periodic behavior of diurnal glucose (e.g., cyclic correlation) may be used. A sliding window (again possibly with temporal weighting) may be used to track changes in health over time. Incorporation of meta-data (e.g., race or body mass index (BMI)), as well as other sensors (e.g., photoplethysmography (PPG)) to make inferences about glycemic health may be used. Categorical classification of subjects (e.g., as nondiabetic, prediabetic, or Type 2 diabetic) may be performed instead of or in addition to calculating a health score. Multiple sensors may be used with the methods disclosed herein to produce a “whole-body” health score. The subject of interest may be classified according to glycemic phenotype. Methods described herein may be applied to calculating health scores for a subject of interest and performing classification of a subject of interest with respect to health conditions other than diabetes, for example, with respect to congestive heart failure. In such an embodiment, distributional data based, for example, on a raw cardiac signal or the RR time series, a graph of the time between beats vs time or beats per minutes vs time, may be used to generate estimated PDFs or CDFs (e.g., using KDE). Calculations described herein may be performed by a processing circuit (e.g., by a central processing unit CPU) connected to memory.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X−Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B. Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1−35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Although exemplary embodiments of a system and method for processing glucose data have been specifically described and illustrated herein, many modifications and variations will be apparent to those skilled in the art. Accordingly, it is to be understood that a system and method for processing glucose data constructed according to principles of this disclosure may be embodied other than as specifically described herein. The invention is also defined in the following claims, and equivalents thereof.

What is claimed is:

1. A method, comprising:
estimating the severity of diabetes in a subject,
the estimating comprising comparing:
distributional glucose data of the subject, and
distributional glucose data of one or more reference subjects.

2. The method of claim 1, wherein the comparing comprises calculating a measure of distance between the distributional glucose data of the subject, and the distributional glucose data of the one or more reference subjects.

3. The method of claim 2, wherein the measure of distance is a Wasserstein distance.

4. The method of claim 2, wherein the measure of distance is a Cramer distance.

5. The method of claim 2, wherein the measure of distance is a Jensen-Shannon distance.

6. The method of claim 1, wherein the distributional glucose data of the subject is based on a plurality of glucose measurements taken at different points in time.

7. The method of claim 6, wherein the distributional glucose data of the subject comprises an estimated probability function of a glucose level of the subject.

8. The method of claim 7, wherein the estimated probability function is a kernel density estimate.

9. The method of claim 7, wherein the glucose level is an interstitial glucose concentration of the subject.

10. The method of claim 7, wherein the glucose level is a blood glucose concentration of the subject.

11. The method of claim 6, wherein the distributional glucose data of the subject is calculated from a set of ordered pairs, each ordered pair comprising a glucose measurement taken at a respective first point in time, and a glucose measurement taken at a point in time separated from the first point in time by a fixed time interval.

12. The method of claim 11, wherein the fixed time interval is within 50% of 60 minutes.

13. The method of claim 11, wherein the distributional glucose data of the subject comprises an estimated multi-variate probability density function of a glucose level of the subject.

14. The method of claim 6, wherein the distributional glucose data of the subject comprises a Fourier transform of the plurality of glucose measurements.

15. The method of claim 1, wherein the one or more reference subjects include a subject diagnosed with prediabetes.

16. The method of claim 1, wherein the one or more reference subjects include a subject diagnosed with Type 2 diabetes.

17. A system, comprising:
a processing circuit; and
memory, operatively connected to the processing circuit and storing instructions that, when executed by the processing circuit, cause the system to perform a method, the method comprising:
estimating the severity of diabetes in a subject,
the estimating comprising comparing:
distributional glucose data of the subject, and
distributional glucose data of one or more reference subjects.

18. The system of claim 17, wherein the comparing comprises calculating a measure of distance between the distributional glucose data of the subject, and the distributional glucose data of the one or more reference subjects.

19. The system of claim 17, wherein the distributional glucose data of the subject is based on a plurality of glucose measurements taken at different points in time.

20. The system of claim 19, wherein the distributional glucose data of the subject comprises an estimated probability function of a glucose level of the subject.
